(19) United States
(12) Patent Application Publication
(10) Pub. No.: US 2004/0203139 A1
(43) Pub. Date: Oct. 14, 2004

(54) Way of using microorganisms for getting energy /bioreactor/

(76) Inventor: David Shtok, Los-Angeles, CA (US)

Correspondence Address: David Shtok, apt. 1, 1434 N. Fuller Av., Los-Angeles, CA, 90046, US

(21) Appl. No.: 10/647,891

(22) Filed: May 26, 2004

Related U.S. Application Data
(60) Provisional application No. 60/454,699, filed on Mar. 17, 2003.

(51) Int. Cl. C12M 1/107

(52) U.S. Cl. 435/289.1; 435/300.1

(57) ABSTRACT

This invention falls into the category of micro biotechnology. The claim is creation of the bioreactor populated with community of microorganisms, which participate in the process of oxygenation of carbohydrates of the natural origin and, as a final result, production of methane gas-CH4. The new bioreactor makes possible the use of methane in automobile and energy production. At the same time it does not create harmful discharge into the atmosphere and keeps the environment 100% free of contamination. This invention applies a unique makeup of microorganisms: producers of methane. Produced methane is pumped through a system of pressure regulators into internal combustion engines, which are used in the energy production and other areas of industry. The bioreactor doesn't have any analogies in the world practice. Its application allows to discontinue the use of oil as the basis for production of gasoline and to replace other energy producing media.

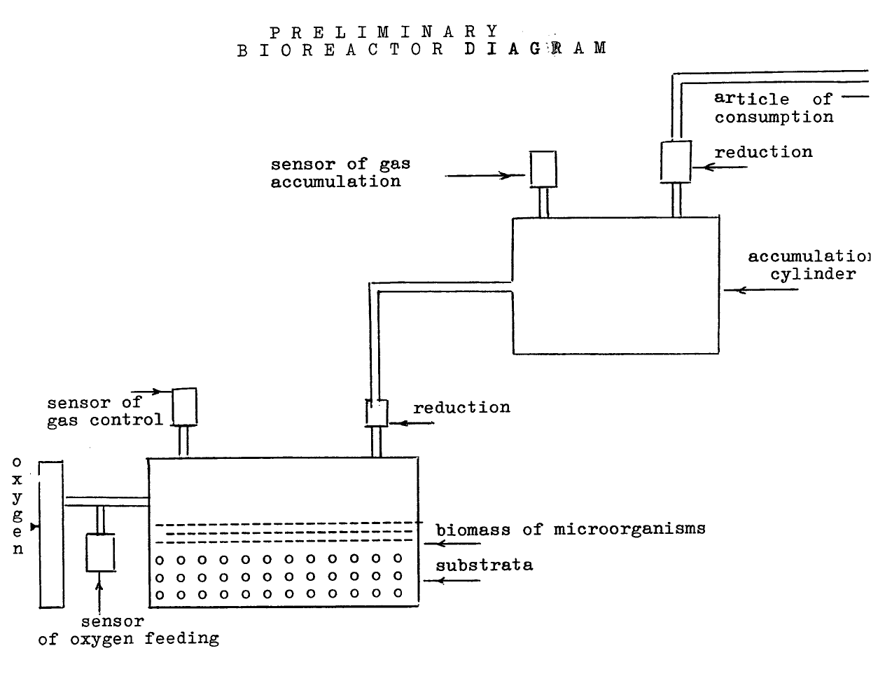

This invention falls into the category of micro biotechnology. It is based on the use of microbiology, biochemistry and genetic engineering. It allows realizing to the fullest extent potential capabilities of the microorganisms in the industry and conservation of the environment.

The goal of this invention is creation of the bioreactor populated with a community of microorganisms participating in the process of oxygenation of carbohydrates of the natural origin and, as a final result, production of methane gas—CH₄.

Methane exists in nature, but its utilization is restricted to the chemical industry and agriculture. The new bioreactor makes possible the use of methane in automobile production, energy production, aviation, military applications and other areas. At the same time it does not create harmful discharge into the atmosphere and keeps the environment 100% free of contamination.

The invention applies a unique makeup of microorganisms consisting of two groups:

1. Producers of methane

2. Regulators of quantity and quality of methane

The new apparatus is based on the cyclic action, which allows control the bioreactor's periodic functioning; this process utilizes the bio-clock principle.

### Microorganisms Functioning in the Reactor

Group one—methane-producing microorganisms.

Methanobacterium formicicum—rods of different shape from elongated to threadlike, with a width of 0.4-0.9 μm. They do not form endo-spores, are motionless and strictly anaerobic. Optimum temperature of their growth is 37-45° C.

Methanosarcina barkerei—spherical in shape with the diameter of 1-1100 μm., do not form endo-spores, are motionless and very strictly anaerobic. Optimum temperature of their growth is 35-40° C.

Methanococcus vannielii—cocci of irregular shape, motionless. They are very strictly anaerobic and have diameter of 1.2-2.2 μm. Optimum temperature of their growth is 60-65° C.

The growth-supporting microenvironment (%) of the methane producing group: NH₄Cl—0.075, K₂HPO₄—0.04, MgCl₂—0.01, CaCo₃—2, C₂H₅OH—1 (ml) in water (Sediment in the water drained from a methane tank).

Group two—Methane oxidizing microorganisms

Methylomonas metanica—rods, either straight, bent or branching. Their size is 0.4-1.1×0.9-4.0 μm.; they are moving, aerobic with the use of oxygen. Optimum temperature of their growth is 20-35° C.

Methylococcus capsulatus—cocci with the diameter of 1.1 μm., motionless, aerobic. Optimum temperature of their growth is 37-45° C.

Methylobacterium organophilum—single rods, sometimes in rosettes, moving 0.8-1.1×1.0-7.8 μm., aerobic. Optimum temperature of their growth is 20-35° C.

The growth-supporting microenvironment for methane oxidizing bacterium is KNO₃—2 g., NaHCO₃—1 g., K₂HPO₄—0.5 g., MgSO₄7H₂O—0.2 g, FeCl₃—traces, distilled water—1 liter.

The seed cultures of microorganisms are produced in small fermenters (5-10 litters); in large quantities the cultures are produced in large fermenters (1,000 liters) at optimum temperatures and in the environments noted above.

Microorganisms used in the reactor were produced by means of genetic engineering and show increased production of methane. They are not toxic to humans and animals.

### THE PRINCIPLE OF THE FUNCTIONING OF THE REACTOR

The growth-supporting environment for the microorganisms in the reactor itself consists of coal, peat, oil refining products and other sources of carbohydrates. Then, the methane-producing bacteria are seeded for the period of 7 days. After that the methane oxidizing bacteria are introduced to prevent forming of critical mass of methane; also oxygen is introduced to activate the oxidizing bacteria and to block the development of the methane producing bacteria. This provides double control of methane production. Different relations with oxygen of methane producing and methane oxidizing bacteria are important factors of the functioning of the reactor. Produced methane is pumped through a system of pressure regulators into internal combustion engines, which are used in the energy production and other areas of industry.

All processes in the reactor are automated and controlled through a system of sensors and controlling devices.

The pilot device with the size of 15×15×15 cm. worked for 15 min. Produced methane was sufficient for bringing to a boiling point of 0.5 liter of water; it used 150 grams of coal dust for the supporting substratum.

The period of functioning of the reactor after a single fuelling could be brought to 30-40 days or more depending of the quantity of the feeding component and the composition of the culture of microorganisms in the reactor.

The bioreactor does not have any analogies in the world practice. Its application allows to discontinue the use of oil as the basis for production of gasoline and to replace other energy producing media.

1. This invention falls into the category of micro biotechnology. The claim is creation of the Bioreactor populated with community of microorganisms, which participate in the process of oxygenation of carbohydrates of the natural origin and, as a final result, production of Methane as—CH₄. The new bioreactor makes possible the use of methane in automobile and energy production. At the same time it does not create harmful discharge into the atmosphere and keeps the environment 100% free of contamination. This invention applies a unique makeup of Microorganisms; producers of Methane. Produced Methane is pumped through a system of pressure regulators into internal combustion engines, which are used in the energy production and other areas of industry. The bioreactor doesn't have any analogies in the world practice. Its application allows to discontinue the use of oil as the basis for production of gasoline and to replace other energy producing media.
